(19) United States
(12) Patent Application Publication
(10) Pub. No.: US 2021/0315444 A1
(43) Pub. Date: Oct. 14, 2021

(54) OPTICAL APPARATUS AND ENDOSCOPE

(71) Applicant: OLYMPUS CORPORATION, Tokyo (JP)

(72) Inventor: Kyosuke Yoshida, Kokubunji-shi, Tokyo (JP)

(73) Assignee: OLYMPUS CORPORATION, Tokyo (JP)

(21) Appl. No.: 17/356,769

(22) Filed: Jun. 24, 2021

Related U.S. Application Data
(63) Continuation of application No. PCT/JP2018/048144, filed on Dec. 27, 2018.

(51) Int. Cl. A61B 1/00; G02B 7/10

(52) U.S. Cl. CPC A61B 1/00188; A61B 1/00193; G02B 7/10

(57) ABSTRACT

An optical apparatus includes an optical system that forms an optical image, a moving frame that holds at least part of the optical system, a fixed frame that encompasses the moving frame and slidably holds the moving frame in an optical axis direction of the optical image, a coil configured to generate a magnetic field to drive the moving frame along the optical axis and at least two magnets disposed at a position where torque with a predetermined moment is generated around an axis parallel to the optical axis by a magnetic force in the moving frame. The at least two magnets include a first magnet and a second magnet that are disposed such that a midpoint of a line connecting respective centers of the first magnet and the second magnet on a cross section orthogonal to the optical axis falls within a cross-sectional region of the moving frame.

### CROSS REFERENCE TO RELATED APPLICATION

This application is a continuation application of PCT/JP2018/048144 filed on Dec. 27, 2018, the entire contents of which are incorporated herein by this reference.

### BACKGROUND OF THE INVENTION

### 1. Field of the Invention

The present invention relates to an optical apparatus that moves back and forth a moving frame including an optical system by a magnetic force to change an optical focal position, and an endoscope.

### 2. Description of the Related Art

Optical apparatuses that move a moving frame including an optical system back and forth in an optical axis direction of the optical system, and can thereby switch a focal position are well known. An image pickup unit provided with such an optical apparatus is provided not only in a camera but also in a communication terminal with a camera, an endoscope and the like.

For example, Japanese Patent Application Laid-Open Publication No. 2017-90504 discloses an endoscope including an optical unit (hereinafter referred to as an “optical apparatus”) provided at a distal end portion of an insertion portion, the optical unit being able to make changeable optical characteristics such as a depth of focus, an image-forming magnification or a view angle with respect to an observation target according to an observation region or a purpose of observation or the like.

### SUMMARY OF THE INVENTION

An optical apparatus according to an aspect of the present invention includes an optical system configured to form an optical image, a moving frame that holds at least part of the optical system, a fixed frame that encompasses the moving frame and slidably holds the moving frame in an optical axis direction of the optical image, a coil configured to generate a magnetic field to drive the moving frame along the optical axis and at least two magnets disposed at a position where torque with a predetermined moment is generated around an axis parallel to the optical axis by a magnetic force in the moving frame, in which the at least two magnets include a first magnet and a second magnet, the first magnet and the second magnet being disposed such that a midpoint of a line connecting respective centers of the first magnet and the second magnet on a cross section orthogonal to the optical axis falls within a cross-sectional region of the moving frame.

An endoscope according to an aspect of the present invention includes an optical apparatus, the optical apparatus including an optical system configured to form an optical image, a moving frame that holds at least part of the optical system, a fixed frame that encompasses the moving frame and slidably holds the moving frame in an optical axis direction of the optical image, a coil configured to generate a magnetic field to drive the moving frame along the optical axis and at least two magnets disposed at a position where torque with a predetermined moment is generated around an axis parallel to the optical axis by a magnetic force in the moving frame, in which the at least two magnets include a first magnet and a second magnet, the first magnet and the second magnet being disposed such that a midpoint of a line connecting respective centers of the first magnet and the second magnet on a cross section orthogonal to the optical axis falls within a cross-sectional region of the moving frame, and an insertion portion including a distal end portion on which the optical apparatus is mounted.

### BRIEF DESCRIPTION OF THE DRAWINGS

FIG. 1 is a diagram illustrating an appearance of an endoscope provided with an optical apparatus according to an aspect of the present invention;

FIG. 2 is a perspective view illustrating a configuration of the optical apparatus of the endoscope according to the aspect of the present invention;

FIG. 3 is a top view illustrating the configuration of the optical apparatus of the endoscope according to the aspect of the present invention;

FIG. 4 is a side view illustrating the configuration of the optical apparatus of the endoscope according to the aspect of the present invention;

FIG. 5 is a front view illustrating the configuration of the optical apparatus of the endoscope according to the aspect of the present invention viewed in an arrow V direction in FIG. 4;

FIG. 6 is a VI-VI cross-sectional view in FIG. 5 of the configuration of the optical apparatus of the endoscope according to the aspect of the present invention:

FIG. 7 is a VII-VII cross-sectional view in FIG. 6 of the configuration of the optical apparatus of the endoscope according to the aspect of the present invention;

FIG. 8 is a cross-sectional view illustrating a configuration of an optical apparatus according to a first modification of the optical apparatus of the endoscope according to the aspect of the present invention;

FIG. 9 is a cross-sectional view illustrating a configuration of an optical apparatus according to a second modification of the optical apparatus of the endoscope according to the aspect of the present invention;

FIG. 10 is a cross-sectional view illustrating a configuration of an optical apparatus according to a third modification of the optical apparatus of the endoscope according to the aspect of the present invention;

FIG. 11 is a cross-sectional view illustrating a configuration of an optical apparatus according to a fourth modification of the optical apparatus of the endoscope according to the aspect of the present invention:

FIG. 12 is a cross-sectional view illustrating a configuration of an optical apparatus according to a fifth modification of the optical apparatus of the endoscope according to the aspect of the present invention; and

FIG. 13 is a cross-sectional view illustrating a configuration of an optical apparatus according to a sixth modification of the optical apparatus of the endoscope according to the aspect of the present invention.

### DETAILED DESCRIPTION OF THE PREFERRED EMBODIMENT

Here, the present invention will be described by taking an endoscope provided with an optical apparatus as an example. Note that the drawings based on each embodiment are schematic ones and a relationship between thickness and width of each component, and thickness ratios or the like among the respective components are different from the actual ones, and some parts may differ from other parts in dimensional relationships or ratios among the drawings.

Although the configuration of the endoscope provided with the optical apparatus will be described hereinafter by taking an endoscope with a flexible insertion portion to be inserted into an upper or lower digestive organ of a living body, a so-called flexible scope, as an example, the present invention is not limited to this, and the present invention is a technique also applicable to a so-called rigid endoscope with a rigid insertion portion used for surgery.

Moreover, the optical apparatus is not limited to one provided for a medical instrument such as an endoscope, but since it is small in size, the present optical apparatus can also be adopted for a mobile phone with a camera, for example.

Hereinafter, the optical apparatus and the endoscope according to an aspect of the present invention will be described based on the accompanying drawings.

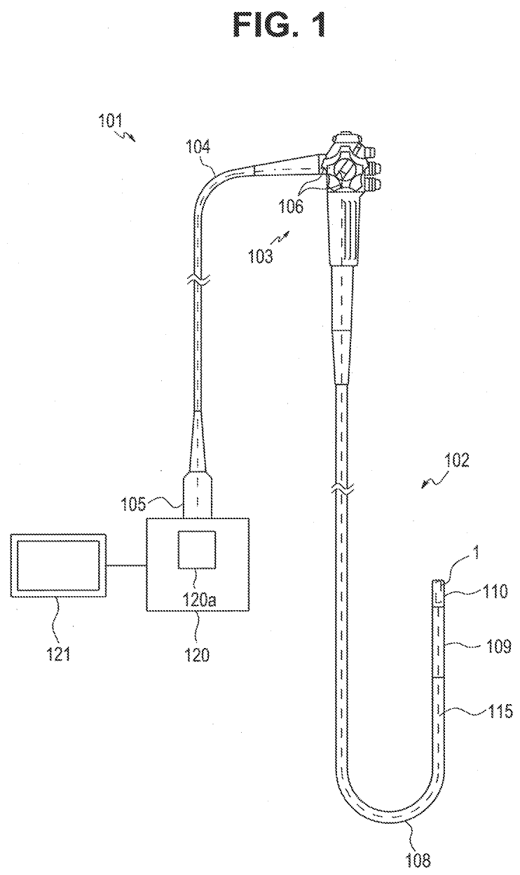

First, an example of a configuration of an endoscope 101 provided with an optical apparatus 1 according to the present invention will be described with reference to FIG. 1.

The endoscope 101 of the present embodiment can be introduced into a subject such as a human body and has a configuration in which an image of a predetermined observation region in the subject is optically picked up.

Note that the subject into which the endoscope 101 is introduced is not limited to a human body, but may be another living body or may be an artificial object such as a machine or a building.

The endoscope 101 mainly includes an insertion portion 102 to be introduced into the subject, an operation portion 103 located at a proximal end of the insertion portion 102, and a universal cord 104 extending from a side of the operation portion 103.

The insertion portion 102 includes a distal end portion 110 disposed at a distal end, a bending portion 109 that is bendable and disposed on a proximal end side of the distal end portion 110 and a flexible tube part 108 disposed on a proximal end side of the bending portion 109 and connected to a distal end side of the operation portion 103, all of which are connected in series.

Although details will be described later, the optical apparatus 1 is provided at the distal end portion 110. Angle operation knobs 106 to operate bending of the bending portion 109 are provided at the operation portion 103.

An endoscope connector 105 to be connected to an external apparatus 120 is provided at a proximal end portion of the universal cord 104. The external apparatus 120 to which the endoscope connector 105 is connected is connected to an image display section 121 such as a monitor via a cable.

The endoscope 101 includes a composite cable 115 inserted through the universal cord 104, the operation portion 103 and the insertion portion 102, and an optical fiber bundle (not illustrated) that transmits illumination light from a light source section provided in the external apparatus 120.

The composite cable 115 is configured to electrically connect the endoscope connector 105 and the optical apparatus 1. When the endoscope connector 105 is connected to the external apparatus 120, the optical apparatus 1 is electrically connected to the external apparatus 120 via the composite cable 115.

Power is supplied from the external apparatus 120 to the optical apparatus 1 and communication between the external apparatus 120 and the optical apparatus 1 is carried out via the composite cable 115.

The external apparatus 120 is provided with an image processing section. The image processing section generates a video signal based on an image pickup device output signal outputted from the optical apparatus 1 and outputs the video signal to the image display section 121. In other words, in the present embodiment, an optical image (endoscope image) picked up by the optical apparatus 1 is displayed on the image display section 121 as a video.

Note that the endoscope 101 is not limited to a configuration in which the endoscope is connected to the external apparatus 120 or the image display section 121, but the endoscope 101 may also be configured to include part of or a whole of an image processing section or a monitor.

A light guide (not illustrated), which is an optical fiber bundle, and which will be described later, is configured to transmit light emitted from the light source section of the external apparatus 120 to an illumination window as an illumination light emission section of the distal end portion 110. Furthermore, the light source section may also have a configuration in which the light source section is disposed in the operation portion 103 or the distal end portion 110 of the endoscope 101.

Here, a configuration of the optical apparatus 1 mounted on the distal end portion 110 of the insertion portion 102 of the endoscope 101 will be described in detail hereinafter.

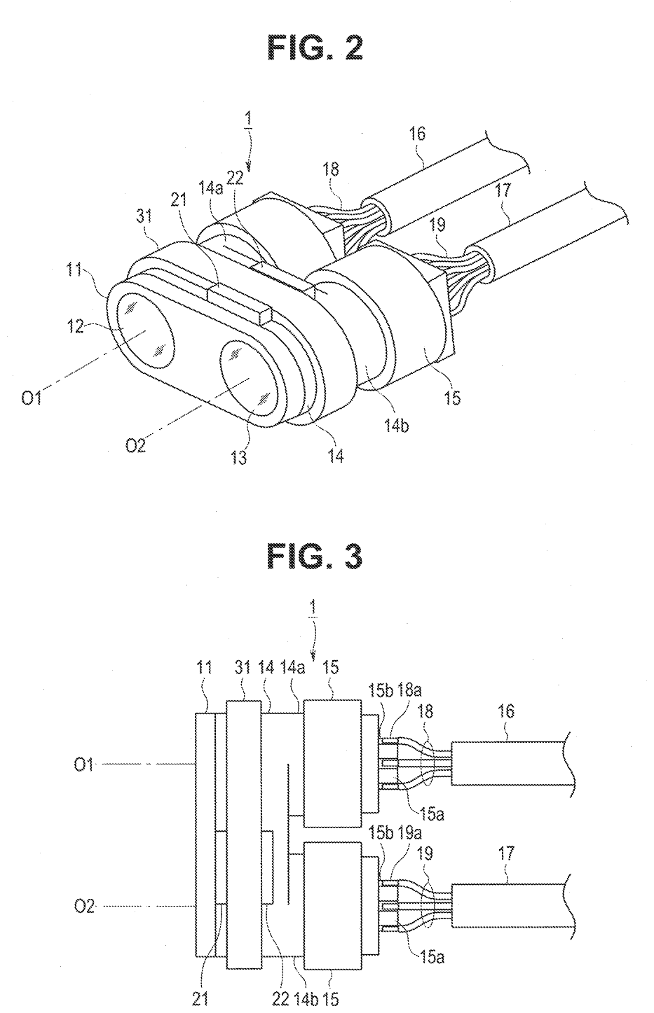

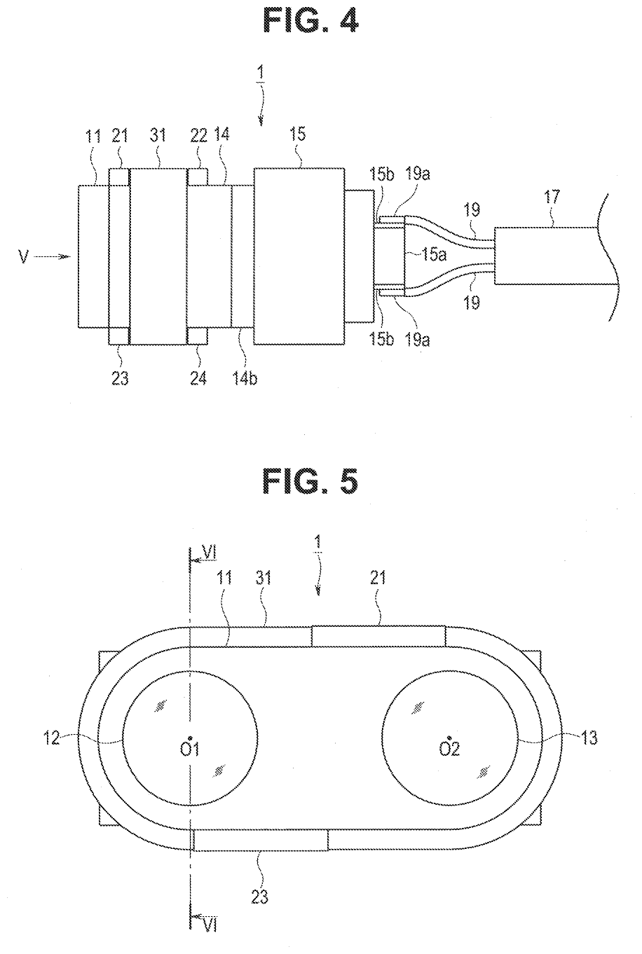

The optical apparatus 1 of the present embodiment illustrated in FIG. 2 to FIG. 5 is a 3D camera that can acquire stereoscopic (3D) images, provided with a stereo optical system that forms two optical images. Note that the optical apparatus 1 including two optical systems that form two optical images is not limited to the 3D camera, but the optical apparatus 1 may have a configuration in which one optical system can perform normal light observation and the other optical system can perform special light observation such as NBI (narrow band imaging).

The optical apparatus 1 includes a lens holding frame 11 having an oval outer cross section, which is a first fixed frame made of a non-magnetic material such as resin or a non-magnetic metal material such as stainless steel configured to hold objective lenses 12 and 13, which are two observation optical systems on the distal end side. Note that the objective lenses 12 and 13 may constitute an observation window exposed at the distal end portion 110 of the endoscope 101.

Shooting light having an optical axis O1 is incident on the objective lens 12. On the other hand, shooting light having an optical axis O2 is incident on the objective lens 13. A parallax between the two shooting lights produces a 3D image.

The lens holding frame 11 is fitted into a guide frame 14 having an oval cylindrical outer cross section on the distal end side, which is a second fixed frame made of a non-magnetic material such as a non-magnetic resin material or a non-magnetic metal material. The guide frame 14 includes two cylindrical sections 14a and 14b having circular cross sections at the proximal end and the two cylindrical sections 14a and 14b are fitted into two substantially bottomed cylindrical image pickup device holding frames 15, which are third fixed frames, made of a non-magnetic resin material such as resin or a non-magnetic material such as a non-magnetic metal material.

A coil 31 wound with fine metal wire such as copper is provided on an outer peripheral portion of the guide frame 14. On the outer peripheral portion of the guide frame 14, a pair of first magnets 21 and 22, and a pair of second magnets 23 and 24 are provided at different substantially linear edge parts in front of and behind the coil 31 so as to sandwich the coil 31.

The two image pickup device holding frames 15 include a rectangular block-shaped wiring connection section 15a at the proximal end portion and a plurality of terminal sections 15b are provided so as to be exposed from the surface of the wiring connection section 15a. Conductors 18a and 19a of a plurality of wires 18 and 19 of image pickup cables 16 and 17 are connected to the plurality of terminal sections 15b by solder or the like.

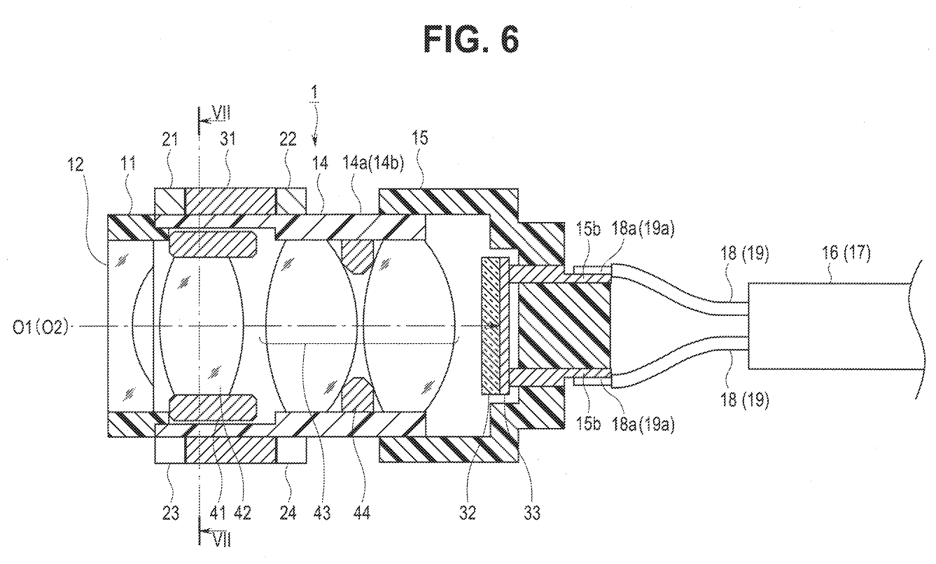

As illustrated in FIG. 6, the guide frame 14 encompasses and accommodates a moving lens frame 41 made of a magnetic material such as iron, nickel or cobalt, the moving lens frame 41 being a sliding member having an oval cross section, so as to be slidable within a predetermined range along the optical axes O1 and O2. The moving lens frame 41 holds two moving lenses 42 in a direction orthogonal to the optical axis O1 (O2).

Note that the moving lens frame 41 made of a magnetic material is caused to slide back and forth according to the direction of a magnetic field generated from the coil 31 by changing an energizing direction of the coil 31.

The optical apparatus 1 of the present embodiment is configured to be able to change the focal position of the subject by driving the moving lens 42 held to the moving lens frame 41 back and forth. Note that optical characteristics of the moving lens 42 according to the drive position may be changed by switching the focus on the subject in the endoscope 101 between near-point and far-point observations or by tele/wide zoom switching.

A fixed lens group 43 composed of two observation optical systems is held in the two cylindrical sections 14a and 14b of the guide frame 14 and a spacer 44 is provided in a space between the lenses in the direction of the optical axis O1 (O2).

An image sensor 33 provided with a cover glass 32 at the front and provided with a solid image pickup device such as a CCD or a CMOS is disposed in each of the two image pickup device holding frames 15. The image sensors 33 are electrically connected to the plurality of terminal sections 15b.

Here, the configuration in which the first magnets 21 and 22 and the second magnets 23 and 24 cause the slidable moving lens frame 41 to move toward one side within the guide frame 14 along the optical axes O1 and O2.

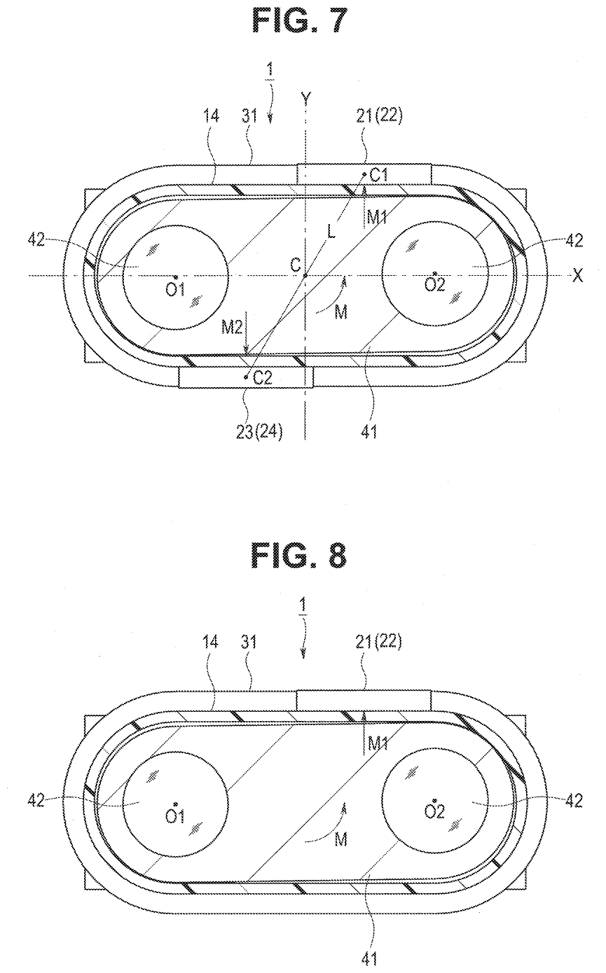

As illustrated in FIG. 7, the first magnets 21 and 22, and the second magnets 23 and 24 are disposed at positions on the outer peripheral portion of the guide frame 14 in a direction orthogonal to the optical axes O1 and O2 and shifted to the left or the right (along an X-axis in FIG. 7) as viewed facing the figure and at positions on either side of the moving lens frame 41 in a vertical direction (along a Y-axis in FIG. 7) as viewed facing the figure.

In other words, the first magnets 21 and 22, and the second magnets 23 and 24 are disposed so as to be located in quadrants in diagonal directions of four quadrants divided by the X-axis orthogonal to the optical axes O1 and O2 in a longitudinal direction and passing through a center point of a cross section of the guide frame 14 and the moving lens frame 41, and the Y-axis orthogonal to the X-axis and in a traverse direction passing through the center point.

Note that a configuration is illustrated here in which the first magnets 21 and 22 are mainly disposed in a first quadrant divided by the X-axis and the Y-axis, and the second magnets 23 and 24 are mainly disposed in a third quadrant divided by the X-axis and the Y-axis. It goes without saying that a configuration in which the first magnets 21 and 22 are disposed in a second quadrant and the second magnets 23 and 24 are disposed in a fourth quadrant may also be adopted.

The moving lens frame 41 in the guide frame 14 always receives an attractive force of the magnetic force M1 of the first magnets 21 and 22, and always receives an attractive force of the magnetic force M2 of the second magnets 23 and 24 in the direction opposite to the magnetic force M1.

In this condition, in the moving lens frame 41, torque with a predetermined moment M is generated around an axis parallel to the optical axes O1 and O2 passing through a midpoint C of a straight line L connecting a center C1 of the first magnets 21 and 22, and a center C2 of the second magnets 23 and 24 on a cross section orthogonal to the optical axes O1 and O2.

Therefore, the moving lens frame 41 is kept in a condition in which an outer surface portion facing the first magnets 21 and 22 remains in contact with an inner surface of the guide frame 14 and an outer surface portion facing the second magnets 23 and 24 remains in contact with an inner surface of the guide frame 14.

By always receiving the rotational moment by the attractive forces of the first magnets 21 and 22 and the second magnets 23 and 24, the moving lens frame 41 is moved to one side and the slide position along the optical axes O1 and O2 is stabilized with respect to a clearance with the guide frame 14.

Note that although the arrangement of the first magnets 21 and 22 and the second magnets 23 and 24 does not matter as long as the midpoint C falls within the cross-sectional region of the moving lens frame 41 so that the torque around the axis parallel to the optical axes O1 and O2 passing through the midpoint C is generated in the moving lens frame 41, it is preferable to arrange the first magnets 21 and 22 and the second magnets 23 and 24 so that the midpoint C would coincide with the cross-sectional center of the moving lens frame 41 to obtain more stable and smooth slidability of the moving lens frame 41.

In other words, it is a preferred configuration of the optical apparatus 1 that the first magnets 21 and 22, and the second magnets 23 and 24 are provided at point-symmetrical positions of the midpoint C. It is further preferable that the magnetic force M1 of the first magnets 21 and 22 be the same as the magnetic force M2 of the second magnets 23 and 24 (M1=M2).

In the optical apparatus 1 in such a configuration, even when reduced in size, sliding operation and reproducibility of the stop position of the moving lens frame 41, which is a sliding portion, become stable. As a result, the optical apparatus 1 demonstrates stable optical performance and can prevent defects such as blur or partial vignetting.

Furthermore, since the magnetic forces M1 and M2 of the first magnets 21 and 22, and the second magnets 23 and 24 keep the optical apparatus 1 in a condition in which a rotational moment around the axis parallel to the optical axes O1 and O2 passing through the midpoint C is always generated in the moving lens frame 41, initial operation of the moving lens frame 41 also becomes uniform during sliding.

With the clearance with the guide frame 14, the moving lens frame 41 does not incline with respect to the optical axes O1 and O2, and so the moving lens frame 41 is not hooked or stopped, and smooth sliding operation is obtained in this way.

As described above, the optical apparatus 1 of the present embodiment is configured to be able to perform sliding operation of the moving lens frame 41 smoothly, respond to miniaturization and high pixel count and stabilize optical performance with high accuracy.

In the case of the optical apparatus 1 that acquires stereoscopic (3D) images in particular, it is possible to prevent partial blur or parallax shift produced at the time of focus adjustment (enlargement operation).

Even when the optical apparatus 1 is miniaturized, it is possible to stabilize optical performance, and thereby downsize the distal end portion 110 of the insertion portion 102 of the endoscope 101 on which the optical apparatus 1 is mounted. As a result, it is possible to reduce the diameter of the insertion portion 102 and also improve insertability of the insertion portion 102 of the endoscope 101 into the subject.

### (First Modification)

The optical apparatus 1 needs only to generate the predetermined moment M, and as illustrated in FIG. 8, the optical apparatus 1 may have a configuration with only the first magnets 21 and 22, without the second magnets 23 and 24.

### (Second Modification)

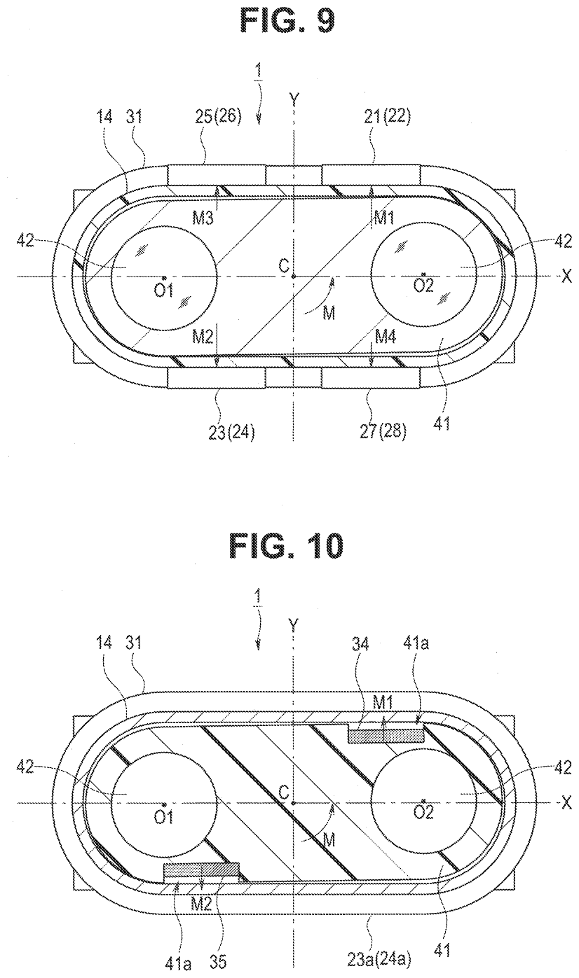

As illustrated in FIG. 9, the optical apparatus 1 may have a configuration in which third magnets 25 and 26 having a magnetic force M3 smaller than the magnetic force M1 of the first magnets 21 and 22 are provided on an outer peripheral portion of the guide frame 14 along the Y-axis with respect to the second magnets 23 and 24, and fourth magnets 27 and 28 having a magnetic force M4 smaller than the magnetic force M2 of the second magnets 23 and 24 are provided on an outer peripheral portion of the guide frame 14 along the Y-axis with respect to the first magnets 21 and 22.

In other words, the optical apparatus 1 has a configuration in which the third magnets 25 and 26 are provided in the fourth quadrant along the X-axis with respect to the first magnets 21 and 22, and the fourth magnets 27 and 28 are provided in the second quadrant along the X-axis with respect to the second magnets 23 and 24.

With such a configuration, the respective magnetic forces M1 and M2 of the first magnets 21 and 22, and the second magnets 23 and 24 are larger than the respective magnetic forces M3 and M4 of the third magnets 25 and 26 and the fourth magnets 27 and 28, and so it is possible to generate the rotational moment in the moving lens frame 41.

It is also possible to adjust the respective magnetic forces M1, M2, M3 and M4 of the first magnets 21 and 22, the second magnets 23 and 24, the third magnets 25 and 26 and the fourth magnets 27 and 28 to cause the moving lens frame 41 to slide smoothly.

### (Third Modification)

The optical apparatus 1 may also have a configuration as illustrated in FIG. 10 in which the guide frame 14 is made of a magnetic material, the moving lens frame 41 is made of a non-magnetic material and a plurality of (here two) magnets 34 and 35 are provided on the moving lens frame 41 side so that torque with the predetermined moment M is generated in the moving lens frame 41.

Note that the moving lens frame 41 is provided with concave portions 41a in which the two magnets 34 and 35 are arranged so that the two magnets 34 and 35 do not directly contact the guide frame 14. In this way, the magnets 34 and 35 do not directly stick to the guide frame 14 and the moving lens frame 41 can slide smoothly.

### (Fourth Modification)

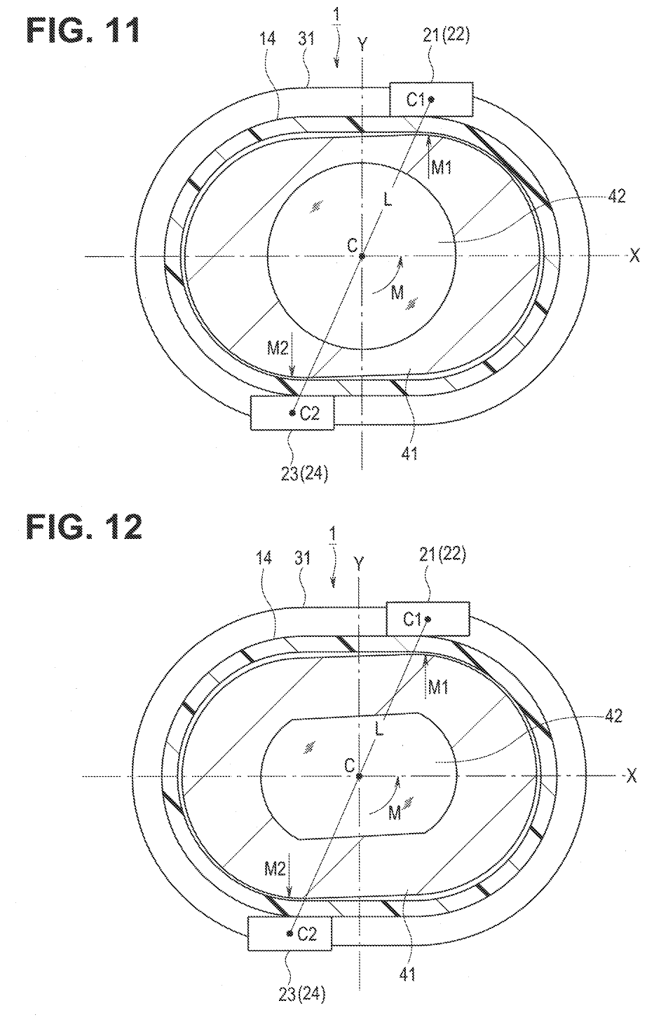

Instead of the configuration of the above-described 3D camera provided with two optical systems (objective lens and moving lens) so as to have parallax and acquire stereoscopic (3D) images, the optical apparatus 1 may also have a configuration with one optical system to acquire one plan view (2D) image as illustrated, for example, in FIG. 11.

Note that the optical apparatus 1 in the present modification has a configuration premised on a cylindrical guide frame 14 having an oval outer cross section and a moving lens frame 41 having an oval outer cross section.

### (Fifth Modification)

Instead of having a perfect circular cross section orthogonal to the optical axis of the optical system in the optical apparatus 1, an optical image formed by the optical system may have an oval shape as illustrated in FIG. 12 so as to be adaptable to a high aspect ratio of 16:9 or above used, for example, in HDTV. Alternatively, an optical image formed by the optical system may have an oval shape adaptable to a high aspect ratio of 16:10 or above so as to be also adaptable to a display monitor with a high aspect ratio of 16:10 or above used in WSXGA+.

Note that the oval optical system is also applicable to a 3D camera that can acquire stereoscopic (3D) images. Note that the oval optical system as illustrated in the present modification is adaptable to the aforementioned oval guide frame 14 and the moving lens frame 41.

Although the moving frame having an oval cross section has been disclosed in the above-described embodiment, the present invention is also effective for an elliptic, which is similar to an oval, or square-shaped moving frame.

### (Sixth Modification)

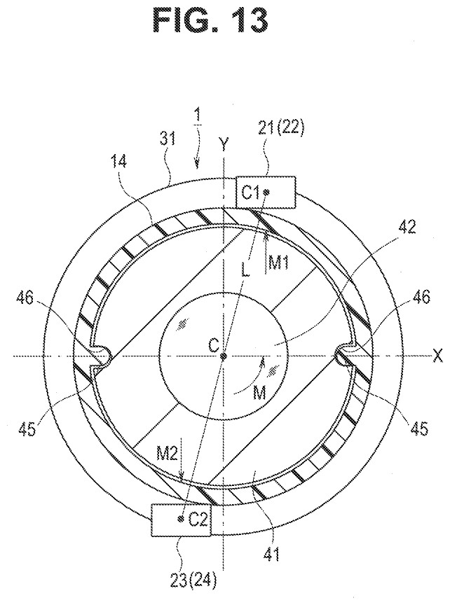

As illustrated in FIG. 13, when the guide frame 14 has a circular cylindrical outer cross section and the moving lens frame 41 has a circular outer cross section, the optical apparatus 1 has a configuration in which the guide frame 14 is provided with a convex portion 45 along the optical axis and the moving lens frame 41 is provided with concave portions 46 with which convex portions 45 engage along the optical axis.

With such a configuration, even when magnetic forces M1 and M2 of the first magnets 21 and 22 and the second magnets 23 and 24 generate torque with the predetermined moment M around an axis parallel to the optical axis O1 passing through the midpoint C in the moving lens frame 41, the engagement of the convex portions 45 with the concave portions 46 restricts rotation of the moving lens frame 41.

Note that by adopting a configuration of reducing frictional resistance of the moving lens frame 41 that slides along the optical axis to the guide frame 14, the optical apparatus 1 described in the above-described embodiment and modifications can be driven with low power consumption.

More specifically, an outer surface of the moving lens frame 41 or an inner surface of the guide frame 14 may be preferably counterbore by cutting such as grooves or may be subjected to “Surface Teflon” processing (registered trademark). Furthermore, it is also possible to reduce power consumption through energizing control of the coil 31 when driving the moving lens frame 41.

Note that a configuration with a voice coil motor (VCM) or the like may be adopted for an actuator to drive the moving lens frame 41.

The inventions described in the above-described embodiment and modifications are not limited to the embodiment and modifications, but various modifications can be made without departing from the spirit and scope of the inventions in the implementation phase. Furthermore, the above-described embodiment and modifications include inventions in various phases and various inventions can be extracted according to appropriate combinations in a plurality of disclosed configuration requirements.

For instance, even when some configuration requirements are deleted from all the configuration requirements illustrated in the embodiment and modifications, configurations from which these configuration requirements are deleted can be extracted as inventions as long as the described problems can be solved and the described effects can be achieved.

What is claimed is:

1. An optical apparatus comprising:
an optical system configured to form an optical image:
a moving frame that holds at least part of the optical system;
a fixed frame that encompasses the moving frame and slidably holds the moving frame in an optical axis direction of the optical image;
a coil configured to generate a magnetic field to drive the moving frame along the optical axis; and
at least two magnets disposed at a position where torque with a predetermined moment is generated around an axis parallel to the optical axis by a magnetic force in the moving frame, wherein
the at least two magnets include a first magnet and a second magnet, the first magnet and the second magnet being disposed such that a midpoint of a line connecting respective centers of the first magnet and the second magnet on a cross section orthogonal to the optical axis falls within a cross-sectional region of the moving frame.

2. The optical apparatus according to claim 1, wherein
the moving frame is made of a magnetic material, and
the magnet is disposed on the fixed frame made of a non-magnetic material.

3. The optical apparatus according to claim 1, wherein
the first magnet and the second magnet are disposed in quadrants in diagonal directions of four quadrants divided by two axes orthogonal to each other and passing through a center point of the moving frame on the cross section.

4. The optical apparatus according to claim 1, wherein the optical system includes two optical systems that form two optical images.

5. The optical apparatus according to claim 1, wherein the optical image formed by the optical system is an optical image with a high aspect ratio.

6. An endoscope comprising:
an optical apparatus comprising:
an optical system configured to form an optical image;
a moving frame that holds at least part of the optical system;
a fixed frame that encompasses the moving frame and slidably holds the moving frame in an optical axis direction of the optical image;
a coil configured to generate a magnetic field to drive the moving frame along the optical axis; and
at least two magnets disposed at a position where torque with a predetermined moment is generated around an axis parallel to the optical axis by a magnetic force in the moving frame, wherein the at least two magnets include a first magnet and a second magnet, the first magnet and the second magnet being disposed such that a midpoint of a line connecting respective centers of the first magnet and the second magnet on a cross section orthogonal to the optical axis falls within a cross-sectional region of the moving frame, and
an insertion portion comprising a distal end portion on which the optical apparatus is mounted.
